(19) United States
(12) Patent Application Publication
(10) Pub. No.: US 2014/0288604 A1
(43) Pub. Date: Sep. 25, 2014

(54) PEDIATRIC APPARATUS, SYSTEM, AND KIT OF COMPONENTS FOR VERTEBRAE STABILIZATION AND CURVATURE CORRECTION, AND METHODS OF MAKING AND USING SAME

(71) Applicant: JAMES H. ALDRIDGE, Augusta, MI (US)

(72) Inventor: JAMES H. ALDRIDGE, Augusta, MI (US)

(21) Appl. No.: 13/850,157

(22) Filed: Mar. 25, 2013

(51) Int. Cl. A61B 17/70

(52) U.S. Cl. CPC A61B 17/7002
USPC 606/264

(57) ABSTRACT

An apparatus, system, and kit of components useful as a stabilization or correction system for a spinal column, especially in the field of pediatrics. A smooth unthreaded rod or a threaded rod is used that can be straight, rigid, bent or flexible depending on the body condition to be addressed. The rod may be bent or flexible to achieve varying degrees of lordosis (backward curvature) or kyphosis (forward curvature) before being affixed to the anchoring means and apparatus. The straightness, curvature, bent, or flexibility of rod depends upon the location along the spinal column. Once installed to the vertebrae, the rod provides the proper, desired curvature and stabilization for the spinal column. Sharp edges and corners of the apparatus are eliminated by rounding the edges and corners.

The present application is a continuation-in-part of and claims priority from U.S. patent application Ser. No. 13/217,195 filed Aug. 24, 2011, which in turn is a continuation-in-part of and claims priority from U.S. patent application Ser. No. 13/005,817 filed Jan. 13, 2011, which in turn is a continuation-in-part of and claims priority from U.S. patent application Ser. No. 12/838,116 filed Jul. 16, 2010, which in turn claims priority from and is based on U.S. Provisional Patent Application 61/288,273 filed Dec. 19, 2009.

### BACKGROUND OF THE INVENTION

The present invention relates generally to a pediatric apparatus, system, and kit of components for stabilizing and/or correcting the curvature of external members, and methods of making and using same.

More particularly, the present invention relates to an apparatus, system, and kit of components for stabilizing and/or correcting the curvature of vertebrae, and methods of making and using same, especially in the field of pediatrics.

The prior, but not necessarily relevant, art is exemplified by: Price, Jr. U.S. Pat. No. 4,048,897; Kies et al. U.S. Pat. No. 6,050,766; Zhao et al. U.S. Pat. No. 7,060,066; and Martinez et al. U.S. Patent Application Publication 2007/0016190.

It is a desideratum of the present invention is to avoid the animadversion, disadvantages and deficiencies of conventional devices and techniques, and to provide a novel apparatus, system and kit of components that eliminates constant, time-consuming, and difficult adjustments.

### SUMMARY OF THE INVENTION

The present invention provides a pediatric kyphosis or lordosis apparatus, comprising: a main central member having rounded edges and a smooth bore therethrough; a rod member for passing through smooth bore of said main central member without contacting said main central member; a predetermined gap between said rod member and said bore; a first generally C-shaped nut member having rounded edges, a partially cylindrical knurled outer surface, and an internally axial bore portion; the first generally C-shaped nut member being removably and selectively connectable to a first end of the main central member; said first nut member and said first end of said main central member are provided with first mating 45-degree surface members; first connection means connecting the first generally C-shaped nut member and the main central member preventing separation thereof; a second generally C-shaped nut member having rounded edges, a partially cylindrical knurled outer surface, and an internally axial bore portion; the second generally C-shaped nut member being removably and selectively connectable to a second end of the main central member; said second nut member and said second end of said main central member are provided with second mating 45-degree surface members; second connection means connecting the second generally C-shaped nut member and the main central member preventing separation thereof; an anchoring device extending from the main central member; and the anchoring device permitting anchoring the apparatus to an external or internal body part member.

The present invention also provides a pediatric kyphosis or lordosis system, comprising, in combination: a first pediatric apparatus including: a first main central member having rounded edges and a smooth bore therethrough; a rod member for passing through smooth bore of said main central member without contacting said main central member; a predetermined gap between said rod member and said bore; a first generally C-shaped nut member having rounded edges, a partially cylindrical knurled outer surface, and an internally axial bore portion; the first generally C-shaped nut member being removably and selectively connectable to a first end of the main central member; said first nut member and said first end of said main central member are provided with first mating 45-degree surface members; first connection means connecting the first generally C-shaped nut member and said first main central member preventing separation thereof; a second generally C-shaped nut member having rounded edges, a partially cylindrical knurled outer surface, and an internally axial bore portion; the second generally C-shaped nut member being removably and selectively connectable to a second end of the first main central member; said second nut member and said second end of said first main central member are provided with second mating 45-degree surface members; second connection means connecting said second generally C-shaped nut member and said first main central member and preventing separation thereof; a first anchoring device rotatably extending from said first main central member; and said first anchoring device permitting anchoring said first pediatric apparatus to an external or internal body part member; a second pediatric apparatus including: a second main central member having rounded edges and a smooth bore therethrough; said rod member passes through said smooth bore of said second main central member without contacting said second main central member; a predetermined gap between said rod member and said bore; a third generally C-shaped nut member having rounded edges, a partially cylindrical knurled outer surface, and an internally axial bore portion; the third generally C-shaped nut member being removably and selectively connectable to a first end of the second main central member; said third nut member and said first end of said second main central member are provided with third mating 45-degree surface members; third connection means axially connecting the third generally C-shaped nut member and the second main central member preventing separation thereof; a fourth generally C-shaped nut member having rounded edges, a partially cylindrical knurled outer surface, and an internally axial bore portion; said fourth generally C-shaped nut member being removably and selectively connectable to a second end of said second main central member; said second nut member and said second end of said second main central member are provided with fourth mating 45-degree surface members; fourth connection means for axially connecting said fourth generally C-shaped nut member and said second main central member to preventing separation thereof; a second anchoring device rotatably extending from the second main central member; and the second anchoring device permitting anchoring the second pediatric apparatus to a second external or internal body part member; whereby the first and second body part members are constrained in a predetermined position and orientation relative to one another.

The present invention also provides a pediatric kyphosis or lordosis kit of components for stabilizing body parts, comprising: a main housing container for holding: two main central members having rounded edges and a smooth bore therethrough; a rod member for passing through said smooth bore of said main central member without contacting said main central member; generally C-shaped nut members having rounded edges, a partially cylindrical knurled outer surface, and an internally axial bore portion; and an anchoring device rotatably extending from each main central member.

An object of the present invention is to provide an apparatus, system and kit of components as described hereinabove which is particularly useful as a stabilization and/or correction system for a spinal column and/or portions thereof, especially in the field of pediatrics.

Another object is to provide an apparatus as described above wherein: said rod member is smooth; and said first and second connection means includes set screws to removably and selectively affix said C-shaped nut members to said smooth rod member.

Another object of the present invention is to provide an apparatus, system and kit of components wherein each nut member is fabricated and designed to threadedly tighten into a locked position by turning same by a predetermined amount which is less than one complete rotation thereof, and to enable the surgeon to determine in which direction the slot of the nut faces, e.g., away from fleshy areas or soft body tissue.

A further object is to provide an apparatus wherein: said rod member has an external thread; and each nut member is fabricated and designed to threadedly tighten into a locked position by turning same by a predetermined amount which is less than one complete rotation thereof.

Another object is provide an apparatus wherein said anchoring device is rotationally connected to the main central member.

A further object is to provide an apparatus wherein said anchoring device is provided with means to threadedly affix said anchoring device to the body part member.

A further object is to provide an apparatus including an O-ring disposed between said rotatable anchoring device and said main central member.

A further object is to provide an apparatus wherein the external thread of said rod member is a radius root control thread.

Other objects, advantages, and features of the present invention will become apparent to those persons skilled in this particular area of technology and to other persons after having been exposed to the present patent application.

### BRIEF DESCRIPTION OF THE DRAWINGS

FIG. 1 is a perspective view of a first embodiment of the apparatus of the present invention.

FIG. 2 is an elevational view of the first embodiment of the system of the present invention.

FIG. 3 is a sectional view taken along the line 3-3 in FIG. 2.

FIG. 4 is a sectional view taken along the line 4-4 in FIG. 3.

FIG. 5 is a sectional view of a second embodiment.

FIG. 6 is a sectional view taken along the line 6-6 in FIG. 5.

FIG. 7 is a profile of the thread of the rod.

FIG. 8 is a top plan view of a kit of components in accordance with the first embodiment.

FIG. 9 is a top plan view of a kit of components in accordance with the second embodiment.

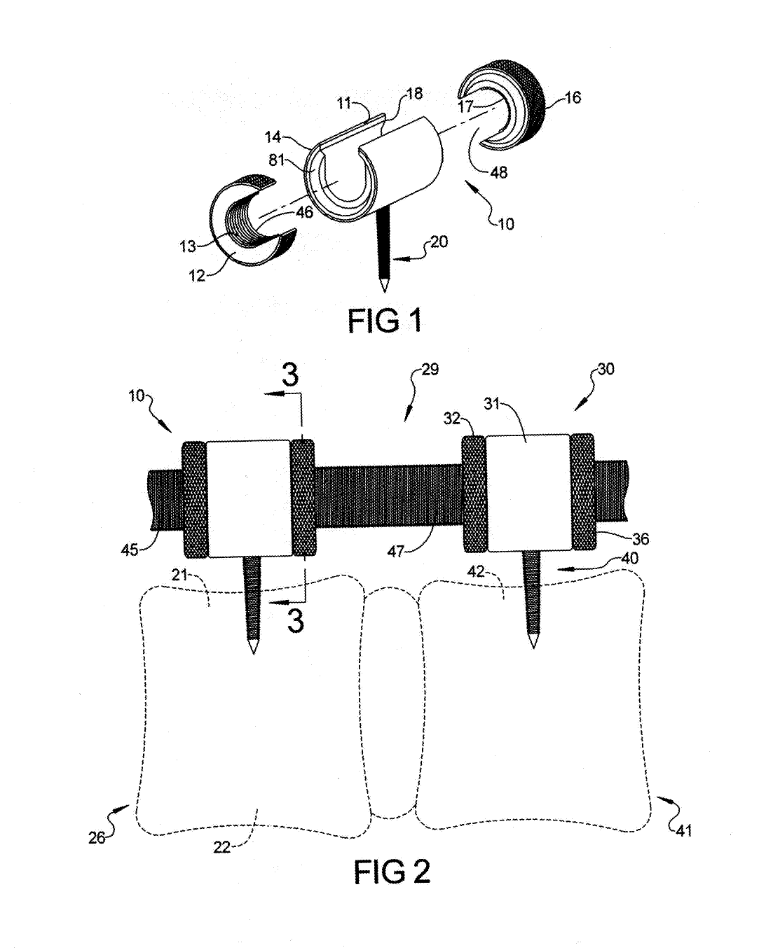

### DETAILED DESCRIPTION OF THE INVENTION

In the drawings and description, similar parts a re designated by like reference numbers.

With reference to the drawings, there is illustrated a first embodiment apparatus 10 including: a main central member or saddle 11 having rounded edges; a first generally C-shaped nut member 12 having rounded edges and a partially-cylindrical knurled outer surface, and an internally axial bore portion 13; and a second generally C-shaped nut member 16 having rounded edges and a partially-cylindrical knurled outer surface, and an internally axial bore portion 17.

The first generally C-shaped nut member 12 is removably attached to a first end 14 of the main central member 11.

Member 12 has a 45-degree surface member 80 which mates with a 45-degree surface member 81 of end 14.

The second generally C-shaped nut member 16 is removably attached to a second end 18 of the main central member 11.

Member 16 has a 45-degree surface member 82 which mates with a 45-degree surface member 83 of end 18.

There is provided a first anchoring device 20 rotatably extending from the main central member 11.

The anchoring device 20 permits anchoring the apparatus 10 to a first external or internal body part member 26, such as a first portion 21 of a vertebrae 22.

With reference to the first embodiment, the nut members 12 and 16 are internally threaded and may individually or together be threaded against the saddle 11.

This causes the members 12 and 16 and saddle 11 to be securely, but removably, axially retained to one another.

With reference to FIG. 2, the system 29 of the invention is illustrated.

System 29 includes the first apparatus 10 and a similar second apparatus 30.

Apparatus 10 has the first anchoring device 20 extending from saddle 11. Device 20 permits anchoring apparatus 10 to the first external or internal body part member 26, such as the first portion 21 of vertebrae 22.

Apparatus 30 has a second main central member or saddle 31 having rounded edges.

A third generally C-shaped nut member 32 has rounded edges, a partially cylindrical knurled outer surface, and an internally axial bore portion.

Member 32 is removably connected to a first end of saddle 31.

A fourth generally C-shaped nut member 36 has rounded edges, a partially cylindrical knurled outer surface, and an internally axial bore portion.

Member 36 is removably connected to a second end of saddle 31.

A second anchoring device 40 rotatably extends from saddle 31. Device 40 permits anchoring the second apparatus 30 to a second external or internal body member 41, such as a second portion 42 of vertebrae 22.

The system 29 constrains the first and second external or internal body members 26 and 41 in a desired predetermined position and orientation relative to one another.

The saddles 11 and 31 and the nuts 12, 16, 32 and 36 are shaped and dimensioned to permit passage therethrough of a rod member 45.

The internally-threaded lock nuts 12, 16, 32 and 36 have internal threads 46 in their respective bore portions to threadedly engage with the thread 47 of rod 45.

Saddles 11 and 31 do not have any threads whatsoever.

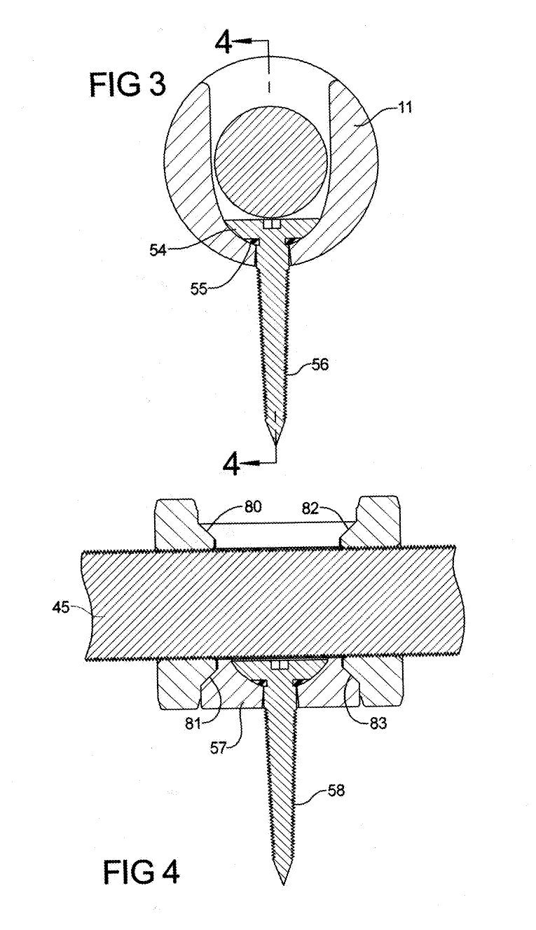

As best seen in FIG. 3, rod 45 does not contact saddle 11 or 31. There is a predetermined clearance between the saddle and the rod 45.

The internally-threaded lock nuts 12, 16, 32 and 36 are shaped and dimensioned to permit passage therethrough of threaded rod member 45, and to permit selective and releasable attachment of the apparatuses 10 and 30 to the rod member 45.

Each internally-threaded lock nut 12, 16, 32 and 36 has a slot 48.

In use, each slot 48 is positioned so that the mouth of the slot 48 is directly adjacent the rod 45 at the desired point of use, whereupon the internally-threaded lock nuts 12, 16, 32 and 36 are moved transversely on rod 45 so that the threads 46 and 47 engage. Thereafter, the internally-threaded lock nuts are rotated through a fraction of a revolution to a locked position. Removal is effected by reversing the above-described sequence.

The apparatuses 10 and 30 can easily be positioned anywhere along the length of rod 45. The rod 45 can easily be removed from the apparatuses 10 and/or 30 or from system 29 for replacement or adjustment.

With reference to FIGS. 3 and 4, the anchoring means 20 or 40 includes an enlarged top portion 54, an O-ring 55, and an elongated threaded member 56. The anchoring means 20 or 40 is rotatably supported in the lower portion 57 of its associated saddle 11 or 31, respectively.

There is also a predetermined clearance between the rod 45 and the uppermost surface of the enlarged top portion 54 of the anchoring means 20 or 40.

The elongated threaded anchoring member 36 is provided with a thread 58, and is used to be selectively and removably threadedly affixed to the external or internal body part member 26 or 41, such as vertebrae portion 21 or 42, respectively.

The O-ring 55 absorbs shocks, provides adjustability, and minimizes the possibility any breakage to other components of the apparatuses 10 or 30 and system 29. Preferably, the O-ring 55 is fabricated from a resilient and/or elastic material.

The internally-threaded lock nuts 12, 16, 32 and 36 hold the saddles 11 and 31, respectively, on the rod 45 in place.

The threads 46 and 47 are provided with radius root control threads to increase their strength and tensile strength.

The apparatuses 10 and 30 and system 29 are particularly useful as a stabilization and/or correction system for a spinal column and/or portions thereof.

The rod 45 can be straight and rigid or somewhat flexible depending on the vertebrae condition to be addressed, and/or may have a predetermined non-straight, curved or bent shape.

For example, the rod 45 may be bent or somewhat flexible to achieve varying degrees of lordosis (backward curvature) or kyphosis (forward curvature) prior to being affixed to the anchoring means and associated apparatus.

Also, the straightness, curvature, bent, and/or flexibility of rod 45 also depends upon the location along the spinal column, i.e., the cervical region may have a kyphotic curve, while the lumbar region may have a lordotic curve. Once installed to the vertebrae, the rod 45 provides the proper, desired curvature and/or stabilization for the spinal column.

Because the apparatus 10 and 30 and system 29 are installed in a patient, the sharp edges and corners of the apparatus are eliminated by rounding all edges and corners, and arranged so that each slot 48 is directed away from soft tissue. For example, when the apparatus is used near the cervical, the closed part of nuts 12, 16, 32 and 36 is arranged to point down to the spine.

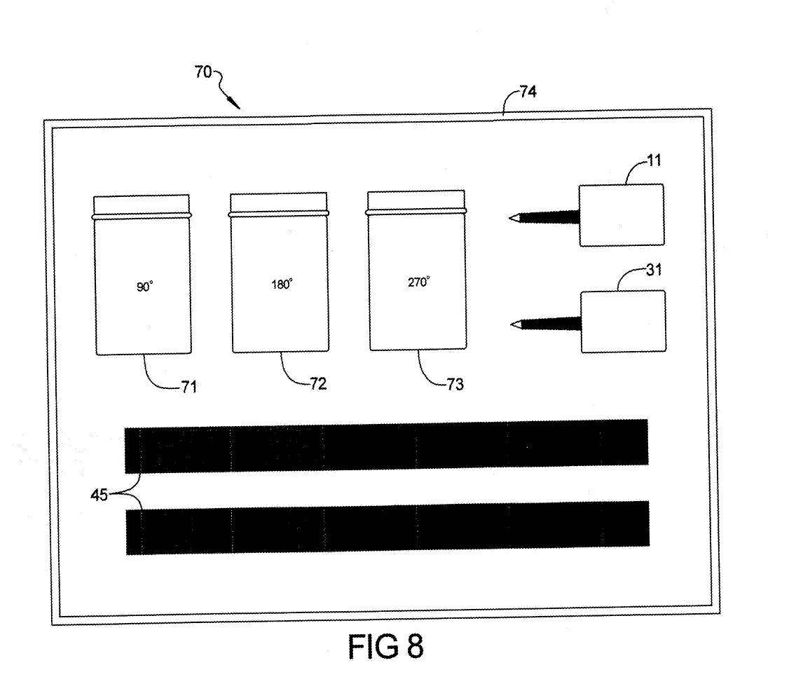

The components of the invention can be sold separately, or in a kit of components 70 illustrated in FIG. 8.

The kit 70 may include a main container 74 for housing the various components.

Some of the nuts can be fabricated to tighten with a 90 degree turn, and can be supplied in a container 71.

Some of the nuts can be fabricated to tighten with a 180 degree turn, and can be supplied in a container 72.

Some of the locknuts can be fabricated to tighten with a 270 degree turn, and can be supplied in a container 73.

The dimensions of the yokes 11 and 31 and the rods 45 can be fabricated to be very small for use in the field of pediatrics.

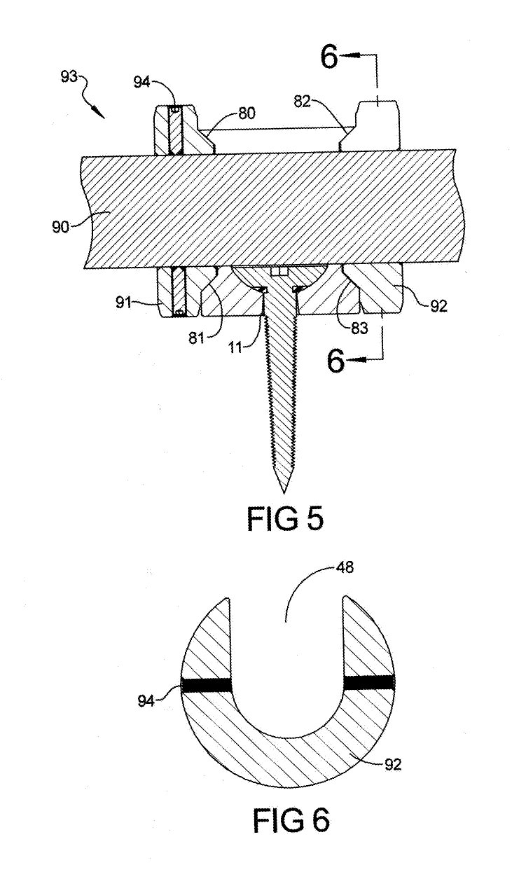

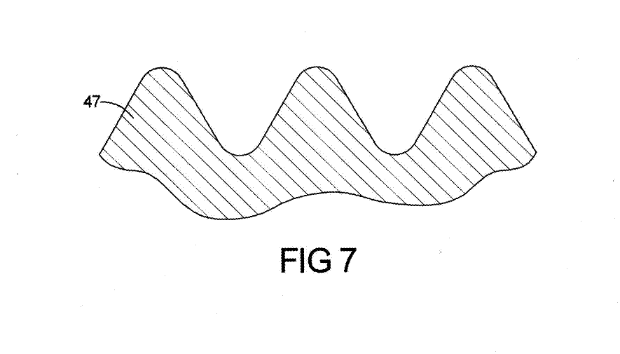

A second embodiment is shown in FIGS. 5, 6 and 9.

In the second embodiment, the rod member 90 is smooth, that is, it is has no threads.

The first generally C-shaped nut member 91 is removably attached to a first end 14 of the main central member 11.

Member 91 has a 45-degree surface member 80 which mates with a 45-degree surface member 81 of end 14.

The second generally C-shaped nut member 92 is removably attached to a second end 18 of the main central member 11.

Member 92 has a 45-degree surface member 82 which mates with a 45-degree surface member 83 of end 18.

The saddle 11 and the nuts 91 and 92 are shaped and dimensioned to permit passage therethrough of the smooth rod member 90.

There is provided a first anchoring device 20 extending from the main central member 11.

The anchoring device 20 permits anchoring the apparatus 93 to a first external or internal body part member 26, such as a first portion 21 of a vertebrae 22.

The nuts 91 and 92 are affixed to rod 90 by set screws 94.

In FIG. 5, the nuts 91 and 92 are disposed 90 degrees relative to one another.

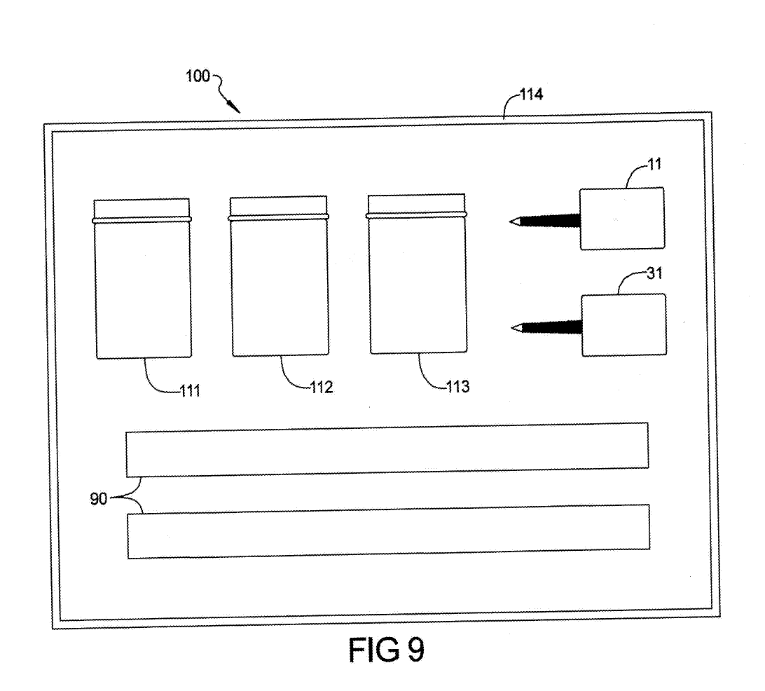

The components of the second embodiment can also be sold separately, or in a kit of components 100 illustrated in FIG. 9.

The kit 100 may include a main container 114 for housing the various components.

The nuts 91 and 92 are housed in a container 111, 112 and 113

The dimensions of the yokes 11 and the smooth rods 90 are fabricated to be very small for use in the field of pediatrics.

There have been described hereinabove only some of the many possible embodiments of the present invention which can be practiced in many different ways.

Many changes, modifications, variations, and other uses and applications will become apparent to those persons skilled in this particular area of technology and to others after having been exposed to the present patent application.

Any and all such changes, modifications, variations and other uses and applications which do not depart from the spirit and scope of the present invention are therefore covered by and embraced within the patent claims set forth hereinbelow.

1. A pediatric kyphosis or lordosis apparatus, comprising:
a main central member having rounded edges and a smooth bore therethrough
a rod member for passing through smooth bore of said main central member without contacting said main central member;
a predetermined gap between said rod member and said bore;
a first generally C-shaped nut member having rounded edges, a partially cylindrical knurled outer surface, and an internally axial bore portion;
the first generally C-shaped nut member being removably and selectively connectable to a first end of the main central member;
said first nut member and said first end of said main central member are provided with first mating 45-degree surface members;
first connection means connecting the first generally C-shaped nut member and the main central member preventing separation thereof;
a second generally C-shaped lock nut member having rounded edges, a partially cylindrical knurled outer surface, and an internally axial bore portion;
the second generally C-shaped lock nut member being removably and selectively connectable to a second end of the main central member;
said second nut member and said second end of said main central member are provided with second mating 45-degree surface members;
second connection means connecting the second generally C-shaped nut member and the main central member preventing separation thereof;
an anchoring device extending from the main central member; and
the anchoring device permitting anchoring the apparatus to an external or internal body part member.

2. The pediatric apparatus of claim 1 wherein:
said rod member is smooth; and
said first and second connection means includes set screws to removably and selectively affix said C-shaped nut members to said smooth rod member.

3. The pediatric apparatus of claim 1 wherein:
said rod member has an external thread; and
each nut member is fabricated and designed to threadedly tighten into a locked position by turning same by a predetermined amount which is less than one complete rotation thereof.

4. The pediatric apparatus of claim 1 wherein
said anchoring device is rotationally connected to the main central member.

5. The pediatric apparatus of claim 2 wherein
said anchoring device is rotationally connected to the main central member.

6. The pediatric apparatus of claim 3 wherein
said anchoring device is rotationally connected to the main central member.

7. The pediatric apparatus of claim 1 wherein
said anchoring device is provided with means to threadedly affix said anchoring device to the body part member.

8. The pediatric apparatus of claim 2 wherein
said anchoring device is provided with means to threadedly affix said anchoring device to the body part member.

9. The pediatric apparatus of claim 3 wherein
said anchoring device is provided with means to threadedly affix said anchoring device to the body part member.

10. The pediatric apparatus of claim 4 wherein
said anchoring device is provided with means to threadedly affix said anchoring device to the body part member.

11. The pediatric apparatus of claim 5 wherein
said anchoring device is provided with means to threadedly affix said anchoring device to the body part member.

12. The pediatric apparatus of claim 6 wherein
said anchoring device is provided with means to threadedly affix said anchoring device to the body part member.

13. The apparatus of claim 1 including
an O-ring disposed between said rotatable anchoring device and said main central member.

14. The apparatus of claim 3 including
an O-ring disposed between said rotatable anchoring device and said main central member; and
wherein said external thread of said rod member is a radius root control thread.

15. A pediatric kyphosis or lordosis system, comprising, in combination:
a first pediatric apparatus having:
a first main central member having rounded edges and a smooth bore therethrough
a rod member for passing through smooth bore of said main central member without contacting said main central member;
a predetermined gap between said rod member and said bore;
a first generally C-shaped nut member having rounded edges, a partially cylindrical knurled outer surface, and an internally axial bore portion;
the first generally C-shaped nut member being removably and selectively connectable to a first end of the main central member;
said first nut member and said first end of said main central member are provided with first mating 45-degree surface members;
first connection means connecting the first generally C-shaped nut member and said first main central member preventing separation thereof;
a second generally C-shaped lock nut member having rounded edges, a partially cylindrical knurled outer surface, and an internally axial bore portion;
the second generally C-shaped lock nut member being removably and selectively connectable to a second end of the main central member;
said second nut member and said second end of said main central member are provided with second mating 45-degree surface members;
second connection means connecting said second generally C-shaped nut member and said main central member preventing separation thereof;
a first anchoring device rotatably extending from said first main central member; and
said first anchoring device permitting anchoring said first pediatric apparatus to an external or internal body part member;
a second pediatric apparatus including:
a second main central member having rounded edges and a smooth bore therethrough
said rod member passes through smooth bore of said second main central member without contacting said second main central member;
a predetermined gap between said rod member and said bore;
a third generally C-shaped nut member having rounded edges, a partially cylindrical knurled outer surface, and an internally axial bore portion;
the third generally C-shaped nut member being removably and selectively connectable to a first end of the second main central member;
said third nut member and said first end of said second main central member are provided with third mating 45-degree surface members;
third connection means axially connecting the third generally C-shaped nut member and the second main central member preventing separation thereof;
a fourth generally C-shaped nut member having rounded edges, a partially cylindrical knurled outer surface, and an internally axial bore portion;
said fourth generally C-shaped nut member being removably and selectively connectable to a second end of said second main central member;
said second nut member and said second end of said second main central member are provided with fourth mating 45-degree surface members;
fourth connection means for axially connecting said fourth generally C-shaped nut member and said second main central member to preventing separation thereof;
a second anchoring device rotatably extending from the second main central member; and
the second anchoring device permitting anchoring the second pediatric apparatus to a second external or internal body part member;
whereby the first and second body part members are constrained in a predetermined position and orientation relative to one another.

16. The pediatric system of claim 15 wherein:
said rod member is smooth; and
set screws removably and selectively affix said C-shaped nut members to said smooth rod member.

17. The pediatric system of claim 15 wherein:
said rod member has an external thread; and
each nut member is fabricated and designed to threadedly tighten into a locked position by turning same by a predetermined amount which is less than one complete rotation thereof.

18. A pediatric kyphosis or lordosis kit of components for stabilizing body parts, comprising:
a main housing container for holding:
two main central members having rounded edges and a smooth bore therethrough;
a rod member for passing through said smooth bore of said main central member without contacting said main central member;
generally C-shaped nut members having rounded edges, a partially cylindrical knurled outer surface, and an internally axial bore portion; and
an anchoring device rotatably extending from each main central member.

19. The pediatric kit claim 18 wherein:
said rod member has an external thread; and
each nut member is fabricated and designed to threadedly tighten into a locked position by turning same by a predetermined amount which is less than one complete rotation thereof.

20. The pediatric kit of claim 18 wherein:
said rod member is smooth; and
set screws removably and selectively affix said C-shaped nut members to said smooth rod member.
